(19) United States
(12) Patent Application Publication
(10) Pub. No.: US 2017/0007697 A1
(43) Pub. Date: Jan. 12, 2017

(54) POTENTIATING IMMUNE RESPONSE AGAINST CANCER

(71) Applicant: Children's Hospital of Orange County, Orange, CA (US)

(72) Inventor: Mustafa Kabeer, Irvine, CA (US)

(21) Appl. No.: 15/276,327

(22) Filed: Sep. 26, 2016

Related U.S. Application Data
(63) Continuation-in-part of application No. 13/973,896, filed on Aug. 22, 2013, now Pat. No. 9,452,206.
(60) Provisional application No. 61/693,180, filed on Aug. 24, 2012.

(51) Int. Cl. A61K 39/39; B02C 23/16; B02C 19/20; A61K 39/09; A61K 45/06

(52) U.S. Cl. CPC A61K 39/39; A61K 39/092; A61K 45/06; B02C 19/20; B02C 23/16; A61K 2039/515

(57) ABSTRACT

A method of potentiating an immune response against a cancer, non-cancerous tumor or both in a patient is provided. The method includes administering splenocytes harvested from all or a part of a patient's spleen. The splenocytes can be administered in combination with a biological response modifier to potentiate an immune response against a cancer, non-cancerous tumor or both in a patient.

### CROSS-REFERENCE TO RELATED APPLICATIONS

This application is a Continuation-In-Part application of U.S. application Ser. No. 13/973,896, filed Aug. 22, 2013, and to be issued as U.S. Pat. No. 9,452,206 on Sep. 27, 2016, which claims the benefit of U.S. Provisional Application No. 61/693,180, filed Aug. 24, 2012, which are hereby incorporated by reference in their entirety.

### BACKGROUND

Field

The embodiments described herein are directed to compositions and methods for potentiating immune responses against a cancer or a non-cancerous tumor or a combination thereof.

Some embodiments described herein are related to potentiating immune responses against a cancer or a non-cancerous tumor or a combination thereof in splenectomized patients.

Some embodiments described herein are related to potentiating immune responses against a cancer or a non-cancerous tumor or a combination thereof in splenectomized patients by administering cells harvested from all or a part of the spleen removed during splenectomy (either partial or total).

Description of the Related Art

There are several medical conditions that often necessitate the removal of all or a part of a patient's spleen, for example as a result of physical trauma, spontaneous rupture, malignancy, or enlargement secondary to conditions such as sickle cell, spherocytosis, thalassemia, malaria, or mononucleosis.

However, patients having undergone splenectomy procedures typically suffer from side effects including reduced immune function and a greater risk of overwhelming post-splenectomy infection (“OPSI”) due to sepsis from encapsulated microorganisms. This is a particular risk for patients whose spleens were removed during childhood. Additionally, patients who have undergone splenectomy may be susceptible to other diseases such as a cancer or a non-cancerous tumor or a combination thereof.

Therefore, it can be rationalized that a possible way of decreasing the risk of OPSI and other post-splenectomy complications and manifestations, such as a cancer or a non-cancerous tumor or a combination thereof, would be to harvest cells from all or a part of the spleen removed during splenectomy, save the harvested cells, and autotransplant the saved cells.

### SUMMARY

In some embodiments, a method of potentiating an immune response against a cancer or a non-cancerous tumor or a combination thereof in a patient in need thereof is provided.

In some embodiments, the method comprises removing all or a part of the patient's spleen, harvesting splenocytes from the removed all or part of the patient's spleen, and administering the harvested splenocytes to the patient, thereby potentiating the immune response against the cancer or the tumor or the combination thereof.

In some embodiments of the method, the immune response against a cancer or a non-cancerous tumor or a combination thereof has been weakened or destroyed in the patient.

In some embodiments of the method, the immune response against a cancer or a non-cancerous tumor or a combination thereof has been weakened or destroyed in the patient by one or more of a chemotherapy, a radiation therapy, a genetic factor, or a co-morbid factor.

In some embodiments of the method, the immune response is cell mediated, humoral or both. In some embodiments of the method, the patient is administered a biological response modifier (BRM) or other immunomodulator or immunotherapy prior to administering the harvested splenocytes, the patient is administered the BRM or other immunomodulator or immunotherapy simultaneously with administering the harvested splenocytes, the patient is administered the BRM or other immunomodulator or immunotherapy after administering the harvested splenocytes, or a combination of the foregoing.

In some embodiments of the method, the BRM is administered orally, subcutaneously, intradermally, intravenously, intramuscularly or intraperitoneal or combination thereof. In some embodiments of the method, the harvested splenocytes are administered intravenously.

In some embodiments of the method, the BRM is an agent that stimulates the patient's immune system against the cancer, the non-cancerous tumor or the combination thereof. In some embodiments of the method, the BRM is an immunomodulatory agent or an immunoadjunctive agent. In some embodiments of the method, the BRM is OK-432.

In some embodiments of the method, the immune response against the cancer or the non-cancerous tumor or the combination thereof is potentiated as measured by an increase in a cellular immunity, a humoral immunity or both against the cancer or the non-cancerous tumor or the combination thereof.

In some embodiments of the method, harvesting comprises homogenizing the part of the patient's spleen and isolating a cellular fraction. In some embodiments of the method, the cellular fraction is preserved for future use.

In some embodiments of the method, the patient is additionally administered chemotherapy, radiation therapy or other immunotherapeutic treatment or any combination thereof. In some embodiments of the method, the patient's cancer or non-cancerous tumor or both has been removed by surgery.

In some embodiments, a device for harvesting cells from all or a part of an organ from a patient is provided. In some embodiments, the device comprises a top portion comprising a lid and a crank comprising a crankshaft rotatably attached to the lid and extending therethrough in a longitudinal direction, wherein the crankshaft further comprises a grinder attached at a distal end of the crankshaft, a bottom portion comprising a filtering component at the proximal end of the bottom portion, and a middle portion defining a cylindrical body, the middle portion configured to be placed between the top portion and the bottom portion, wherein the top, middle, and bottom portions are joined together to define a cavity therein, and wherein the grinder is configured to grind all or a part of the organ from the patient placed within the cavity.

In some embodiments, the device further comprises a handle attached to the crank. In some embodiments of the device, the crank is attached to a motor. In some embodiments, the device comprises a port configured to be connected to a fluid source.

In some embodiments of the device, the lid comprises one or more channels in fluidic communication with the port and the cavity to permit the fluid from the port to enter the cavity.

In some embodiments of the device, the grinder implement comprises a grinding surface provided with one or more cutting channels. In some embodiments of the device, at least one of the top portion and middle portion, or the middle portion and bottom portion, may be secured together by threads.

In some embodiments of the device, the filtering component comprises a sieve or a membrane filter. In some embodiments of the device, a distal end of the bottom portion is configured to attach to a collection device. In some embodiments of the device, the organ is spleen.

In some embodiments, a method of obtaining a population of splenocytes is provided. In some embodiments, the method comprises inserting all or part of a spleen into a device for harvesting cells from all or a part of an organ from a patient, wherein the device comprises a cavity configured to receive all or a part of the organ, the cavity comprising a grinder implement at a proximal end of the cavity and a filtering component at a distal end of the cavity, grinding all or a part of the organ by pressing and rotating the grinder implement against the tissue, supplying fluid into the cavity, filtering the ground all or a part of the organ and fluid through the filtering component, and collecting the ground all or a part of the organ and fluid in a receptacle.

In some embodiments, the method of obtaining a population of splenocytes comprises harvesting splenocytes from the collected all or a part of the organ and fluid.

### BRIEF DESCRIPTION OF THE DRAWINGS

FIG. 1A illustrates a perspective view of an embodiment of a tissue grinding device.

FIG. 1B illustrates a perspective view of an embodiment of a tissue grinding device.

FIG. 2A illustrates a cross-sectional view of an embodiment of a tissue grinding device.

FIG. 2B illustrates a cross-sectional view of an embodiment of a tissue grinding device.

FIG. 3A illustrates a cross-sectional view of an embodiment of a tissue grinding device connected to a fluid source.

FIG. 3B illustrates a side view of an embodiment of a tissue grinding device connected to a fluid source.

FIG. 4 illustrates the underside of a lid of an embodiment of a tissue grinding device.

FIG. 5 illustrates the underside of an embodiment of a grinder implement.

FIG. 6 is an illustration along the proximal end of an embodiment of a bottom portion of a tissue grinding device.

FIG. 7 is an illustration of the distal end of an embodiment of a bottom portion of a tissue grinding device.

FIG. 8 is a graph of antibody titers after subcutaneous vaccination with Streptococcus pneumoniae polysaccharide antigen.

FIG. 9 is a graph of basal antibody titers without exposure to antigen.

FIG. 10 is a graph of antibody titers after intravenous administration of killed Streptococcus pneumoniae cells.

### DETAILED DESCRIPTION

The structure of the spleen is thought to be an integral part involved in immune functionality. The vascular flow through the sinusoids allows antigenic debris to be presented to the resident reticuloendothelial cells lining the sinuses. These cells in turn can present processed antigen to activate other cells within the immune system. It has therefore long been felt that this milieu of the spleen must be preserved in order to maintain function. The spleen also serves as a filter within the body, for example, for intravascular bacterial contaminants.

In addition to the physical structure and location of cells to allow for adequate antigen recognition, there are specific populations of cells such as the lymphocytes that have innate ability and function irrespective of the structure of the spleen that allow them to contribute to both humoral and cell mediated immune responses. These cells are important in that they may possess learned or developed antigen memory that would otherwise be lost with the removal of these cell populations.

As such, surgical treatment focuses on conserving the spleen if possible, especially when treating pediatric patients. If a splenectomy is required, an attempt may be made to preserve at least a portion of the spleen via a partial splenectomy. However, some cases still necessitate a complete splenectomy. In this regard, there are suggestions that splenectomy may ablate immune responses against pathogens, cancers and/or non-cancerous tumors.

Accordingly, numerous studies have examined the feasibility of autologous splenic transplantation as a possible alternative in unsalvageable cases requiring splenectomy. Improved antibody responses along with increased levels of opsonins and tuftsin have been observed in some autologous splenic transplantation experimental models.

Certain studies have shown that in order to achieve any benefit in humoral immunity, at least approximately half of the spleen should be retained. Some studies reported that good outcomes resulted from autotransplants within the mesentery in comparison to intramuscular transplants. Additionally, some studies have shown that improved antibody titers were obtained with intraperitoneal autotransplantation of splenic tissue. Further studies have shown an increased survival rate with a 50% splenectomy compared to a total splenectomy, in a mouse model, when exposed to a Streptococcal challenge.

However, splenic autotransplantation is not frequently practiced because of complications such as autotransplant fibrosis, aseptic necrosis, or bowel adhesion and/or obstruction. Such complications may necessitate further surgery. Additionally, there is some experimental evidence of a lack of efficacy of such autotransplants, as certain studies have shown that autotransplanted spleens have been found to undergo approximately 8% necrosis each year, and are therefore soon below optimal efficacy.

Aside from function, it has also been noted that transplanted spleen sections have been found to have decreased size of peri-arteriolar lymphatic sheath (“PALS”) along with changes in density of B cell, macrophages and T-cell ratios. Not only have changes in parenchymal architecture been observed, but vasculature may also be altered by dilation of vessels in the marginal zone, pulp cords and red pulp where antigen presentation occurs in the spleen.

As stated above, the risks of OPSI are elevated in all splenectomized patients, and the most frequent causes of OPSI include encapsulated organisms, specifically Streptococcus pneumoniae. Currently, vaccine and antibiotic prophylaxis are used to help prevent OPSI in splenectomized patients. Although polyvalent pneumococcal vaccines are available and used for prophylaxis in cases of necessary splenectomy, there are cases where patients have still succumbed to fatal sepsis due to OPSI. Additionally, some studies have shown that serum titers against certain pneumococcal subtypes decline over time to nonprotective levels. Rather, although vaccination is most effective if given prior to splenectomy, this is not always possible.

In some embodiments, the present disclosure are related to the isolation, delivery and autotransplantation of cells, such as splenocytes and lymphocytes, to patients, in particular patients undergoing a splenectomy (either partial or total). When administered to a patient, some embodiments described herein may increase the patient's immunity and reduce the likelihood of a post-splenectomy infection.

Embodiments disclosed herein relate to the isolating and delivering lymphocytes, in particular splenic lymphocytes and splenocytes, to patients requiring or desiring to strengthen their immune system and other potential benefits such as decreased risk of Type II Diabetes, risk of death from cardiac events, and even obesity. In particular, for patients undergoing a splenectomy, some embodiments may serve to reduce the likelihood of a post-splenectomy infection and other such complications.

It will be understood by one of ordinary skill in the art that although certain portions of the description herein may refer to animals such as mice, the embodiments described herein may be used in a non-limiting sense in humans and other animals as well. Non-limiting examples are companion animals and animals of commercial importance such as dogs, cats, guinea pigs, hamsters, rats, sheep, cattle, chicken, turkey, etc.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the inventions herein described.

After splenectomy, a patient typically suffers a reduced immunity resulting from a loss of the immune functionality provided by the spleen. Spleen loss may lead not only to a reduced cell-mediated immune response (through T-lymphocytes and other macrophages), but will also lead to a reduced humoral immune response due to a loss of B-lymphocytes, antibodies produced by these B-lymphocytes, and the loss of spleen structure functioning in antigen presentation and removal.

Thus, splenectomy may lead to reduced immune response not only because of the loss of splenic structure in mediating the immune response, but also due to the loss of the significant population of splenocytes and other constituents, including lymphocytes, that are present therein. These lymphocytes may include, but are not limited to, B cells (including plasma B cells, memory B cells, and follicular B cells), T cells (including cytotoxic T cells, memory T cells, helper T cells, natural killer T cells, suppressor T cells, and gamma delta T cells), natural killer cells, and progenitor lymphopoietic stem cells.

Accordingly, splenocytes and/or lymphocytes and other immune-boosting constituents may be isolated from a patient and reintroduced to the patient to boost the patient's immune response. Preferably, splenocytes and/or lymphocytes from the spleen are reintroduced to the patient. While non-autologous or xenotranfused splenocytes and/or lymphocytes may be used (while accounting for immunologic rejection mechanisms), preferably the splenocytes and/or lymphocytes are autologous and therefore the source of splenocytes and/or lymphocytes is the patient's own spleen.

Cells may be extracted from any tissue by using embodiments of the devices and methods discussed below. In some embodiments, the tissue may be a part of an organ or an entire organ. In some embodiments, cells may part of the tissue, for example, lymphocytes. In some embodiments, the cell are splenocytes when the tissue is the spleen or portions thereof. Of course, it will be understood by one of ordinary skill in the art that the device provided herein can be used for tissue homogenization and/or extraction of cells other than lymphocytes or from sources other than splenic tissue.

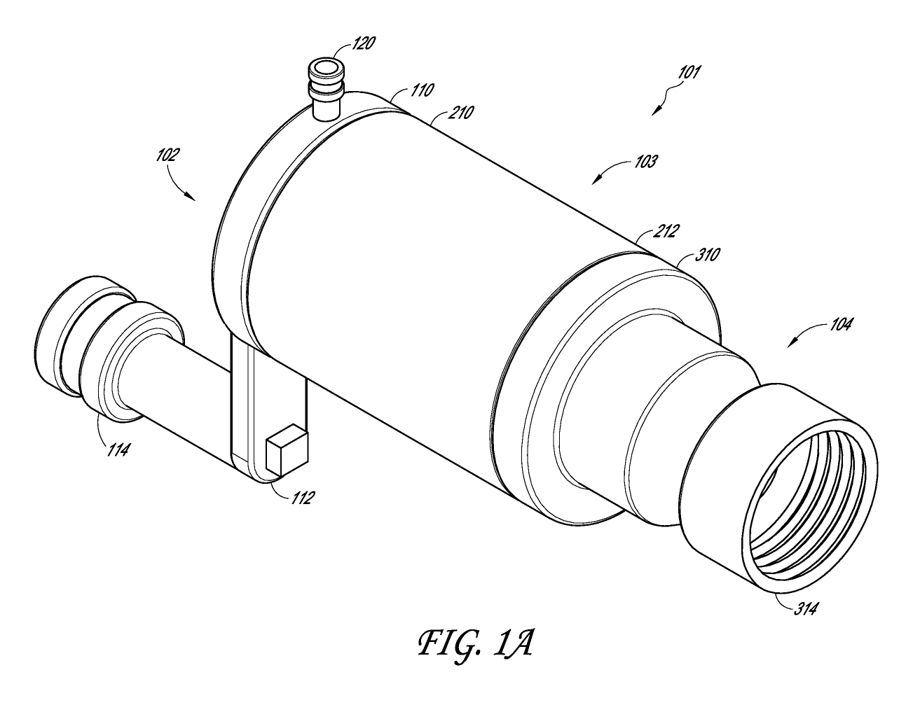

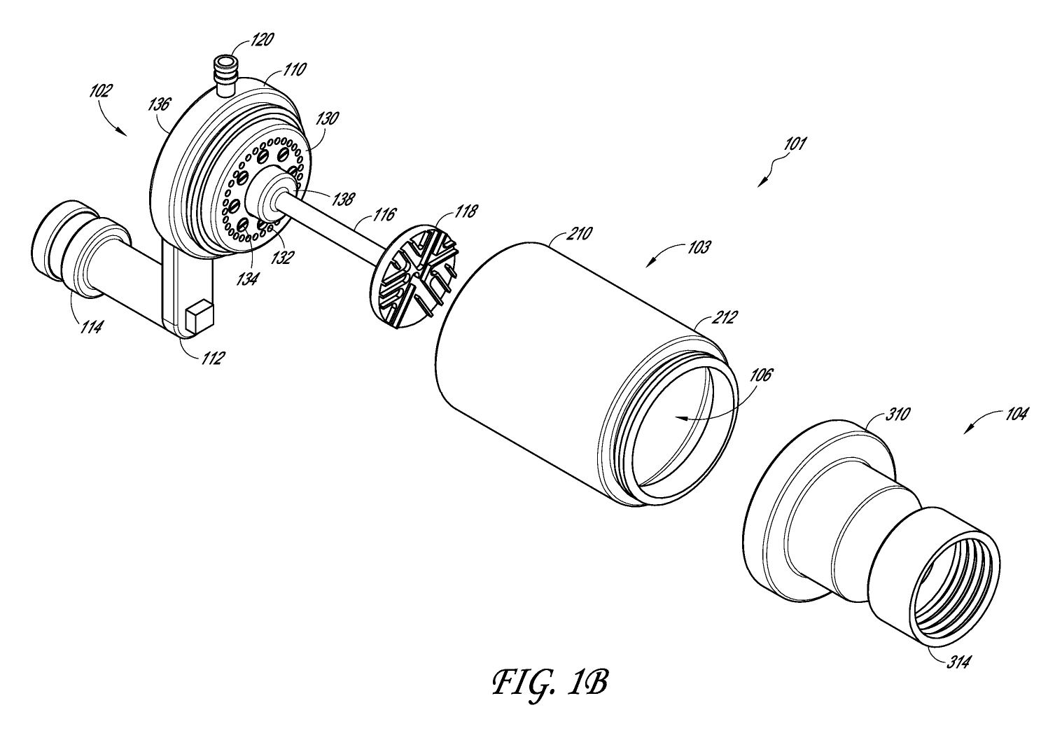

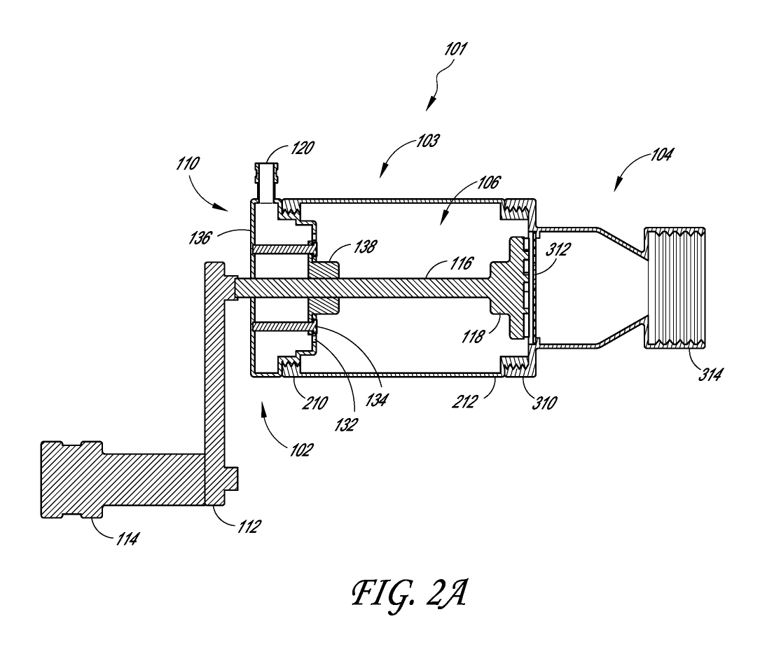

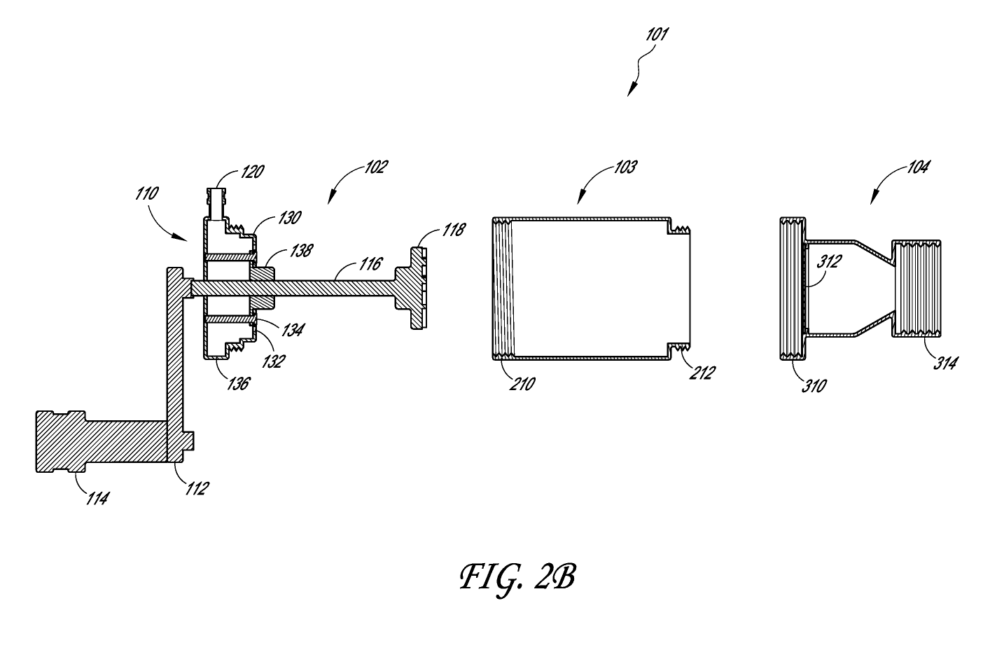

FIG. 1A illustrates a perspective view of an embodiment of a tissue grinding device 101, while FIG. 1B illustrates an exploded view of the same device 101. Cross-sectioned views of an embodiment of the device 101 are also illustrated in FIG. 2A and FIG. 2B. The device 101 may be used to isolate splenocytes from spleen or lymphocytes from a tissue, in particular splenic tissue. The tissue grinding device 101 may be constructed from multiple pieces configured to attach or mate to each other, and in one embodiment comprises a top portion 102, a middle portion 103, and a bottom portion 104. In some embodiments, however, the device 101 may be constructed as a single unit.

Preferably, the device 101 is constructed from a sterilizable material such as metal or plastic. In some embodiments, the device 101 is constructed so as to be reusable, which may be beneficial, for example, if the device 101 is to be used in developing countries where access to disposable devices is difficult.

In some embodiments, it may be preferable for the device 101 to be constructed from a metal such as steel or aluminum that may be autoclaved or otherwise sterilized for reuse. In other embodiments, the device 101 may be constructed so that all or part of the device may be disposed after use. Such embodiments may thus only require that the device 101 be durable enough for a single use, and it may in some embodiments be constructed at least in part from a plastic.

The top portion 102 comprises a lid 110, which is preferably configured to attach, join, or mate with a proximal end 210 of the middle portion 103, and may comprise threads or latches configured to secure both parts together. When the top portion 102, middle portion 130, and bottom portion 104 are attached together, they define an interior cavity 106, which is preferably dimensioned to accept a tissue sample. As discussed herein, the tissue sample may be all or part of a spleen removed from a patient.

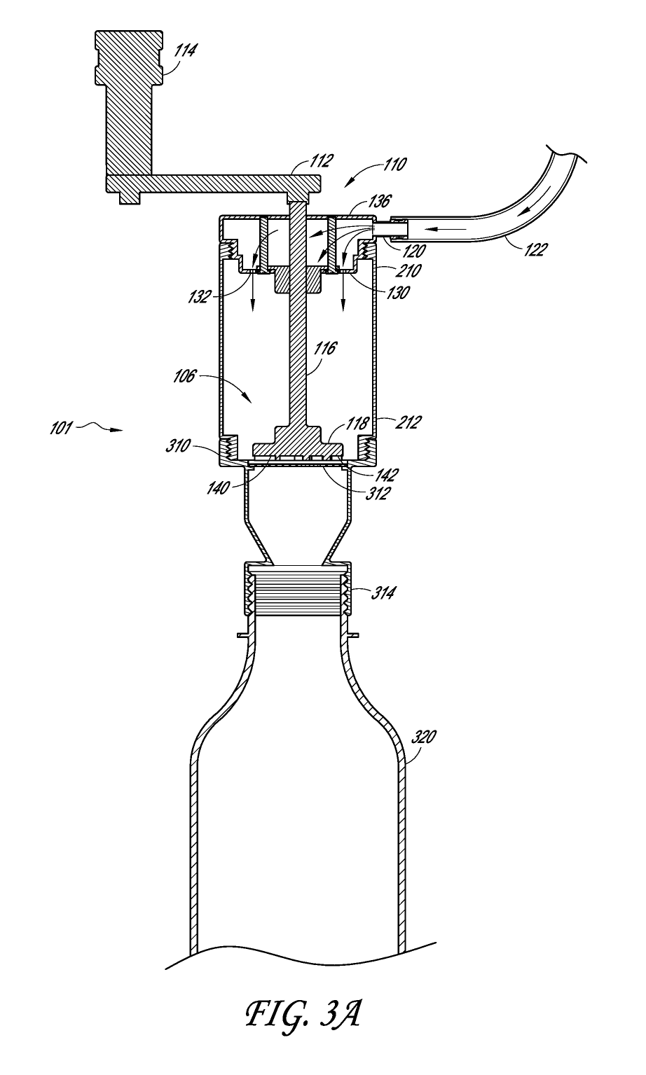

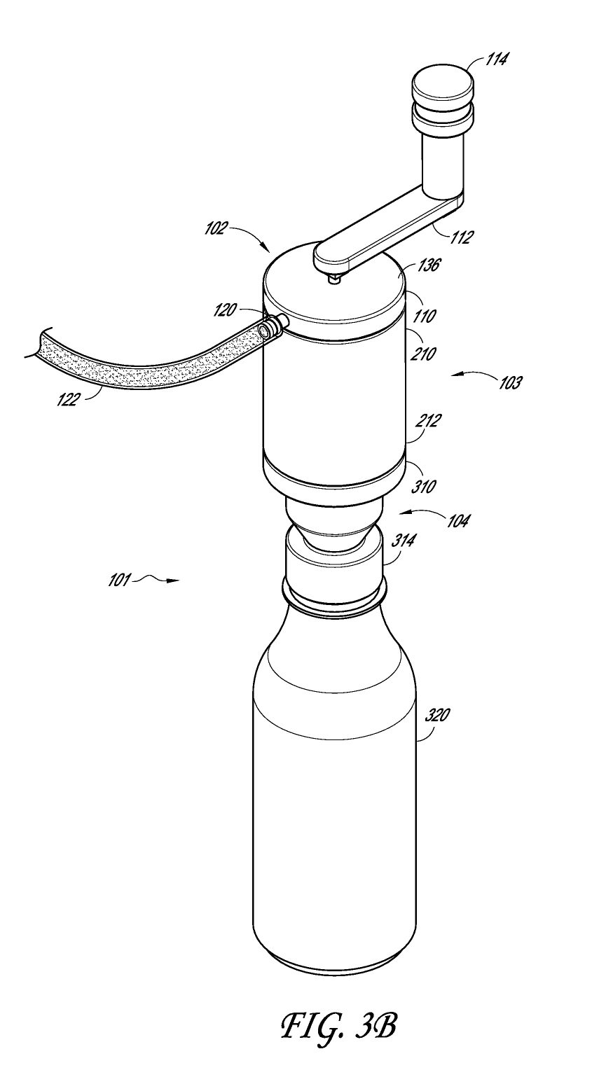

A crank 112 is preferably rotatably attached to the lid 110. In a preferred embodiment, the crank 112 is provided with a handle 114 configured to permit an operator to turn the crank 112, but in some embodiments all or part of the handle 114 may be replaced with a motor or such device capable of rotating the crank 112. The crank 112 is rotatably attached to a proximal end of a crankshaft 116, which extends in a longitudinal direction through the lid 110. A grinder implement 118 is preferably attached at the distal end of the crankshaft 116, and will be discussed further below in relation to FIG. 3. Because the grinder implement 118 rotates, the cavity 106 is preferably cylindrical.

Similarly as with the top portion 102 and middle portion 103, the distal end 212 of the middle portion 103 and a proximal end 310 of the bottom portion 103 are preferably configured to attach, join, or mate together, for example, using threads or latches. A filtering component 312 such as a sieve is preferably provided in the bottom portion 104, and provides a surface for the grinder implement 118 to push against a tissue or a piece thereof placed within the cavity 106. A distal end 314 of the bottom portion 104 is preferably configured to attach to or be received into a receptacle 320 to receive a tissue slurry.

As illustrated in FIG. 3A and FIG. 3B, a port 120 may be provided on the lid 110, for example a top lid portion 136, so as to permit a fluid such as a wash solution, a buffer (e.g., saline, phosphate buffer saline (“PBS”)) or a buffered medium to be supplied therethrough, for example via a supply conduit 122.

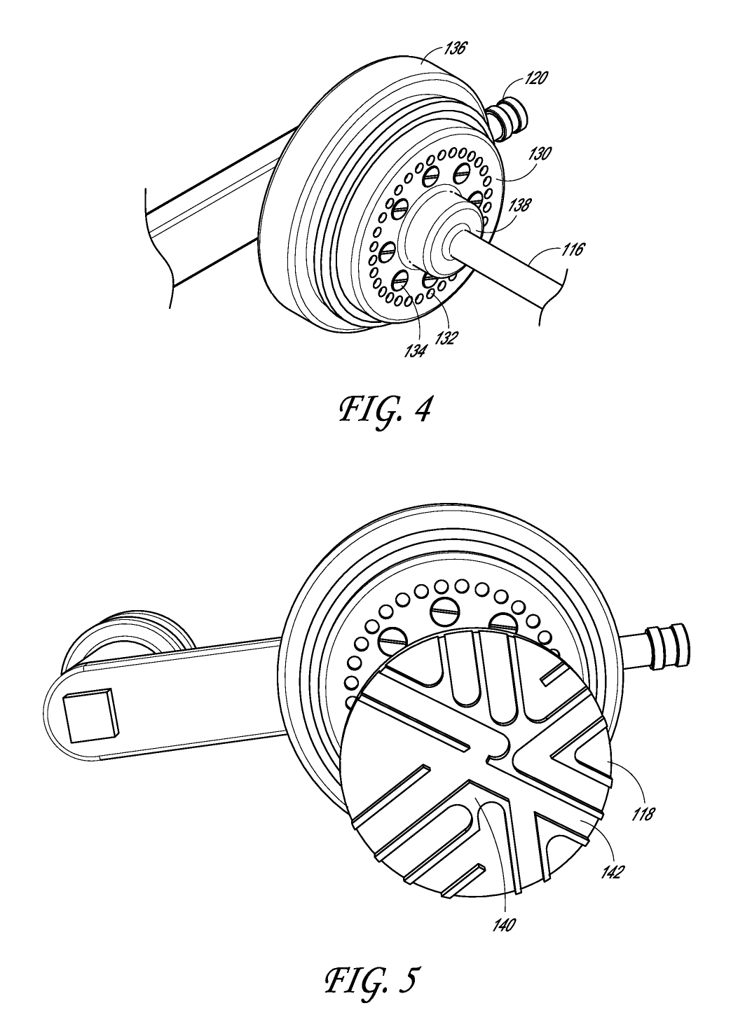

In a preferred embodiment, the fluid is normal saline, as described below in relation to Example 1. This fluid may help flush or move tissue out of the cavity 106. This aspect will be described in further detail below in relation to FIG. 4. Preferably, the receptacle 320, which may be, for example, a sterile bottle, is secured to the distal end 314 of the bottom portion 104. Of course, the port 120 may not necessarily be provided on the lid 110, but may be for example be provided onto the middle portion 103 or bottom portion 104.

FIG. 4 illustrates a close-up view of an embodiment of an underside 130 of the lid 110. In some embodiments, the underside 130 is provided with multiple channels 132 to allow the fluid supplied via the port 120 to enter into the cavity 106. Screws 134 are shown here serving as fixation mechanisms to attach the underside 103 to the lid 136. Of course, the underside 130 may be attached using other means, such as welding or riveting.

With reference now to FIG. 5, an embodiment of a grinder implement 118 is illustrated as viewed from a distal end. In some embodiments, the grinding surface 140 of the grinder implement 118 is preferably configured to press and grind tissue placed within the cavity 106 against the filtering component 312 in the bottom portion 104. Preferably, in some embodiments, the grinder implement 118 is of a slightly smaller diameter than the cavity 106 so as to permit fluid entering the cavity 106 (for example via the channels 132) to reach the tissue sample. In some embodiments, the grinder implement 118 may be provided with one or more through channels that can permit fluid to reach the tissue being ground by the grinding surface 140.

In some embodiment, the grinding surface 140 preferably comprises one or more cutting channels 142 which may be formed, for example via machining, to form sharp edges that can cut, grind, or macerate tissue. In some embodiments, blades or other cutting implements may also be attached to the grinding surface 140. In some embodiments, and as illustrated here, the one or more cutting channels 142 abut with the outer perimeter of the grinder 118, and as such allow the fluid supplied via the channels 132 to reach a greater portion of the tissue sample being ground.

Preferably, in some embodiments, the grinder implement 118, or at least grinding surface 140, is provided with cutting channels 142 or other cutting implements that are constructed from a suitably hard and durable material such as a metal, ceramic, or hardened plastic that may cut, grind, or macerate tissue without being substantially dulled, weakened, or broken while doing so. Additionally, in some embodiments, these should be suitably hard and durable in conjunction with any grinding media, such as grinding beads or grit, that may be used.

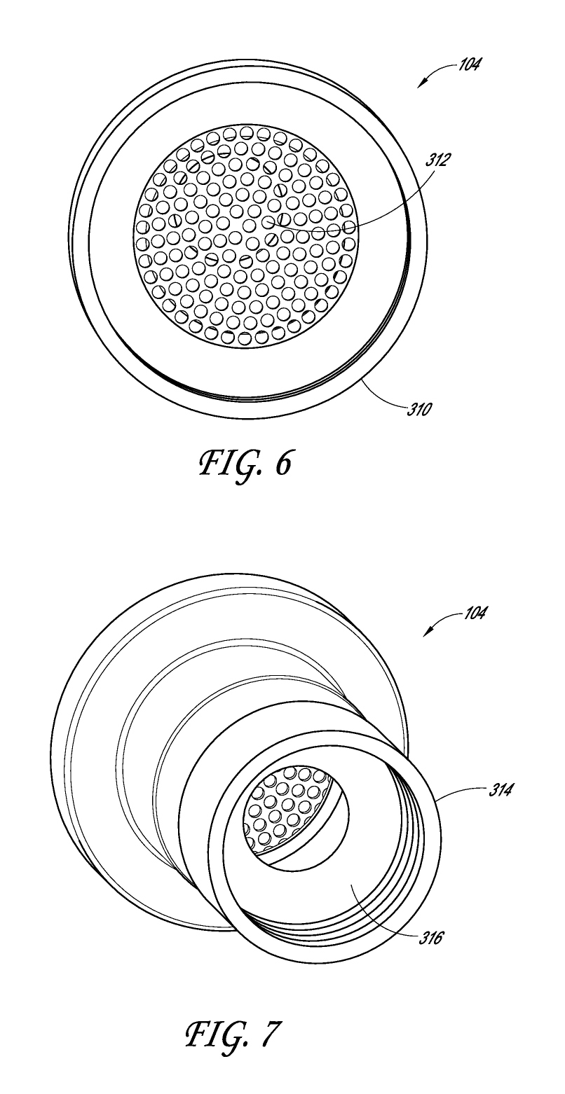

FIG. 6 illustrates a view along the proximal end 310 of the bottom portion 104 of an embodiment. The proximal end 310 is configured to mate with the distal end 212 of the middle portion 103 (not illustrated here), and may comprise threads or other securement mechanisms disposed thereon. The filtering component 312 may be attached, for example, along its outer perimeter, to an inner wall of the bottom portion 104. In some embodiments, however, the filtering component 312 may be attached to the middle portion 103.

In some embodiments, the filtering component 312 may provide multiple functions. First, it preferably acts as a solid surface for the grinder implement 118 to press against the tissue sample inserted into the cavity 106. Second, it is preferably provided with one or more apertures that are small enough to prevent fibrous components from the tissue sample from passing through it, while being large enough to permit cells such as splenocytes and/or lymphocytes to pass through.

In some embodiments, the filtering component may have apertures measuring between 500 μm to 5 mm. In some embodiments, the apertures measure preferably 2 mm to 4 mm. In some embodiments, the apertures measure even more preferably 3 mm. In some embodiments, the apertures are circular. In some embodiments, the apertures are of other shapes.

In some embodiments, the filtering component 312 is removable, and in other embodiments the filtering component 312 is fixed or permanently attached to the body of the bottom portion 104, for example via welding. In some embodiments, in particular those provided with a removable filtering component 312, the filtering component 312 may comprise multiple sub-components. For example, a first filtering component 312a may be mechanically stronger and be provided with larger apertures under which a second filtering component 312b may be situated.

The second filtering component 312b could for example be less mechanically strong and able to withstand pressure from the grinder implement g, thus requiring that it be positioned under a stronger first filtering component 312a, but may be provided with smaller apertures to promote more efficient filtering. The second filtering component 312b, could, in some embodiments, be a membrane filter. In some embodiments, the filtering component 312 may comprise other attachments or devices for separating cells such as lymphocytes from a homogenized tissue medium, including flow cytometry (including fluorescence-activated cell sorting (“FACS”)), and affinity purification (e.g., via antibodies).

FIG. 7 illustrates a view along the distal end 314 of the bottom portion 104. This view illustrates the attachment mechanism for attaching the bottom portion 104 to the collection device 320. Preferably, the attachment mechanism comprises threads configured to secure to the collection device 320. In some embodiments, the collection device 320 is a sterile bottle, for example the bottle illustrated in FIG. 3A and FIG. 3B. A sealing mechanism, such as an o-ring 316 may also be provided to prevent leakage of fluids in the junction between the attachment mechanism and the collection device 320.

In use, the device 101 may be assembled substantially as illustrated in FIG. 1A and FIG. 1B. A tissue sample, such as all or part of a spleen, may then inserted into the cavity 106, which is accessible when the top portion 102 is removed from the middle portion 103. Optionally, grinding media may be added to the cavity 106. The top portion 102 is then replaced over the middle portion 103 and secured thereto. The crankshaft 116 is then moved in a longitudinal direction downward until the grinder implement 118 makes contact with the tissue sample, thereby pressing it against the filtering component 312. The crank 112 and/or crankshaft 116 may then be rotated, for example via the handle 114 (or using a motor attached to either the crank 112 or the crankshaft 116), thereby cutting and grinding the tissue within the cavity 106.

In some embodiments, optionally, fluid such as a wash solution or buffer may be supplied via a conduit 122 attached to the port 120. This fluid exits the top portion 102 via the channels 132 so as to enter into the cavity 106 and to the tissue being ground. Macerated tissue then passes through the filtering component 312 for collection into a receptacle such as the receptacle 320. As previously mentioned, other assays may also be performed onto the cell extract, including flow cytometry and purification of other extracted cellular constituents.

### EXAMPLES

The examples described below illustrate non-limiting experiments conducted to demonstrate the efficacy of lymphocyte reinfusion after splenectomy.

### Example 1

### Animal Study Splenectomy

Balb/C infant female mice (Sasco, Omaha, Nebr.) weighing 20-25 grams were acquired for the purposes of the example. The mice were free from pathogens and kept in filter isolation throughout the course of study. They were housed in an accredited animal care facility at the CHOC Children's Hospital Research Institute Vivarium, and placed on a routine photoperiod with a regular temperature and given laboratory chow and water, all under standard guidelines.

The mice (excluding the control group) were then selected for splenectomy. The operative procedures were performed after the animal was induced with 2% halothane anesthesia and oxygen, and maintained on a constant flow of oxygen with 0.80% halothane delivered via a nose cone.

The abdomens of the mice were cleansed with Betadine solution and shaved. A midline abdominal incision was made, bringing the spleen into the field of view. The spleen was then removed, after cauterizing attached vessels, and placed in a sterile solution of PBS (NaCl 8 g/L, KCl 0.2 g/L, NaHPO₄ 1.15 g/L, KHPO₄ 0.2 g/L, pH 7.2) and kept on ice. The control group had the spleen mobilized and placed back into position without any other disruption. Incisional wounds were closed in two layers and further secured with surgical skin suture.

### Example 2

### Isolation of Splenic Lymphocytes

The spleens removed from the mice in Example 1 were then cut into 2 mm×2 mm squares and placed on a cell sorter sieve made from steel wire mesh and together placed over a 60×15 petri dish. Using a circular grinding motion, the pieces were pressed against the screen with the plunger of a 10 ml. syringe using PBS for irrigation until mostly fibrous tissue remained on the screen. Of course, it will be recognized that the tissue grinding device illustrated above in relation to FIG. 1-FIG. 7 may preferably be used.

The solution was then centrifuged for ten minutes in a Beckman rotor at 1500 rpm, and the supernatant discarded. The pellet was resuspended in 10 ml of ACK lysing buffer (NH₄Cl 8.29 g/L, KHCO₄ 1 g/L, Na₄EDTA 37.2 mg/L, pH 7.2) and incubated for five minutes at room temperature with occasional shaking. Another 10 ml of PBS was added and the solution was centrifuged again for ten minutes at 1500 rpm, followed by discarding of the supernatant. The resulting pellet was washed in PBS twice, each time centrifuging for ten minutes at 1500 rpm.

Finally, the pellet was resuspended in a 5 ml PBS, placed onto 20 ml of Ficoll/Paque gradient (Pharmacia LKB, Piscataway, N.J.) and centrifuged for 20 minutes at 1500 rpm with brakes off. The interphase layer was then aspirated out and washed in PBS (pH 7.22) three times. A cell sample stained with Trypan blue was placed on a haemocytometer and examined under the microscope for purity, viability, and counts.

### Example 2A

### Isolation of Splenocytes

The spleens removed from the mice in Example 1 were then cut into 2 mm×2 mm squares and placed on a cell sorter sieve made from steel wire mesh and together placed over a 60×15 petri dish. Using a circular grinding motion, the pieces were pressed against the screen with the plunger of a 10 ml syringe using PBS for irrigation until mostly fibrous tissue remained on the screen. Of course, it will be recognized that the tissue grinding device illustrated above in relation to FIG. 1-FIG. 7 may preferably be used.

The solution was then centrifuged for ten minutes in a Beckman rotor at 1500 rpm, and the supernatant discarded. The pellet was resuspended in 10 ml of ACK lysing buffer (NH₄Cl 8.29 g/L, KHCO₄ 1 g/L, Na₄EDTA 37.2 mg/L, pH 7.2) and incubated for five minutes at room temperature with occasional shaking. Another 10 ml of PBS was added and the solution was centrifuged again for ten minutes at 1500 rpm, followed by discarding of the supernatant. The resulting pellet was washed in PBS twice, each time centrifuging for ten minutes at 1500 rpm.

Finally, the pellet was resuspended in a 5 ml PBS. A cell sample stained with Trypan blue was placed on a haemocytometer and examined under the microscope for purity, viability, and counts.

### Example 3

### Preparation and Inoculation with Nonviable Streptococcus pneumoniae Cells

Streptococcus pneumoniae Type III cells were purchased from ATCC (Bethesda, Md.). These cells were inoculated into tryptic soy broth (Difco Labs Detroit, Mich.), previously adjusted to pH 7.7, and grown for 4-6 hours at 37° C. Formaldehyde was added to a concentration of 0.1% and the cell suspension was stored at 4° C. after being washed three times in PBS (pH 7.2). Prior to use, the cultured cells were washed three times in sterile PBS and centrifuged into a pellet. They were also test plated onto chocolate agar to ensure nonviability and tested for presence of a capsule with an India ink stain and Quellung positive with Pneumococcus type III specific antisera (Difco, Detroit, Mich.).

The mice from Example 1 were then immunized with 1×10³ of the prepared nonviable Streptococcus pneumoniae cells intravenously, using McFarland nepholometry for quantification.

### Example 4

### Antibody Measurements

Polysaccharide antigens were coupled to protein for adsorption in accordance with the procedure set forth by Gray (Gray, B., ELISA methodology for polysaccharide antigens: Protein coupling of polysaccharides for adsorption to plastic tubes, Journal of Immunological Methods, 28: 187-19.2, 1979).

Briefly, three test tubes A-C were prepared such that tube A had 0.5 ml of 0.05 N NaOH with 0.001% phenolpthalein, tube B had 1 mg of cyanuric chloride crystals, and tube C had 0.1 ml of 0.2% poly-1-lysine (MW 54,000, Sigma Chemicals, St. Louis, Mo.). A polysaccharide (Pnuimmune, Lederle Labs, Pearl River, N.Y.) solution of 100 μl (2.5 mg/ml) was alkalinized for 10 sec by swirling in tube A.

Activation was then accomplished by pouring the contents of tube A into tube B and swirled the contents for ten seconds, at which point the solution turned colorless. The test tube contents were then coupled to poly-1-lysine in tube C and refrigerated at 4° C. for 2 hrs. Coupled polysaccharide was diluted in a 1:4 ratio in PBS (pH 7.2) and eventually used for adsorption onto enzyme-linked immunosorbent assay (“ELISA”) plates to test for antibody titers as described below.

Blood was then obtained from each mouse in Example 1 via retro-orbital venous plexus, prior to immunization, and every seven days after immunization for six weeks. Sera was separated from the blood samples and stored at −20° C.

Antibody titers against Streptococcus pneumoniae polysaccharide were quantified by ELISA. ELISA plates were coated with 50 μl. of pneumococcal polysaccharide vaccine coupled to poly-1-lysine. Plates were then incubated at 37° C. for two hours and then washed three times in PBS with 0.5% Tween. All free sites on the plate were blocked using PBS-Tween (0.5%)-Gelatin (1%) and incubated at 37° C. for two hours. The plates were rinsed again 3× in PBS-Tween (0.5%). Subsequently, 50 μl of mouse antiserum (diluted 1/1000) was added to each well and incubated overnight at 4° C.

All wells were again washed three times in PBS-Tween (0.05%). Next, 50 μl of a 1/100 dilution of goat anti-mouse Ig antisera linked to alkaline phosphatase was added to each well and incubated at 37° C. for one hour. All wells were rinsed three times in PBS-Tween (0.05%). Finally, 50 μl of p-nitrophenol phosphate (1 mg/ml) (Sigma Chemicals, St. Louis, Mo.) in diethanolamine buffer (pH 9.6) was added to each well and incubated for 30 min. prior to reading on an ELISA reader (Dynatech).

### Example 5

### Analysis and Results

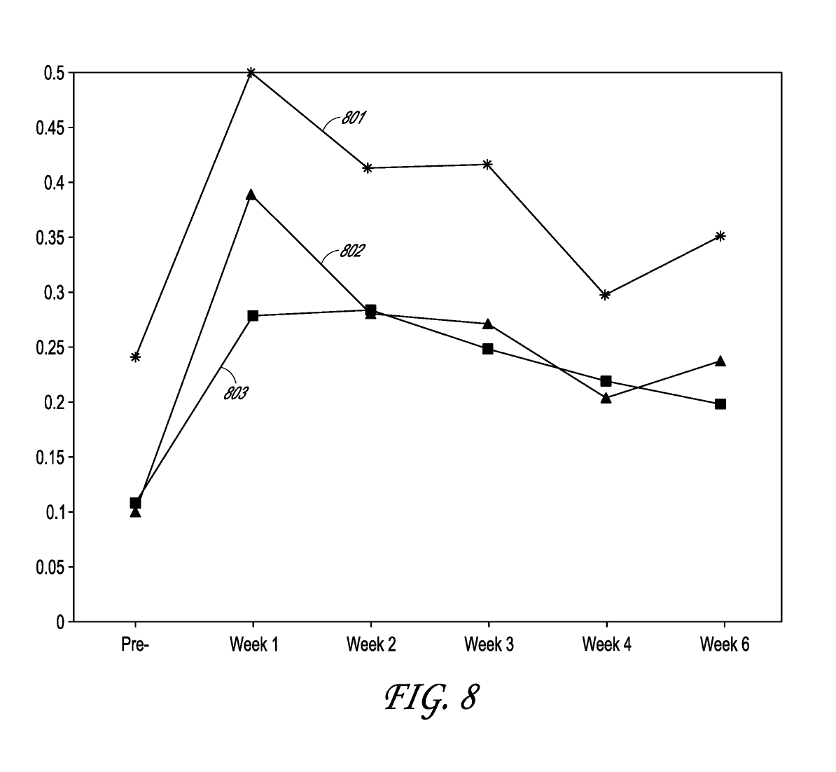

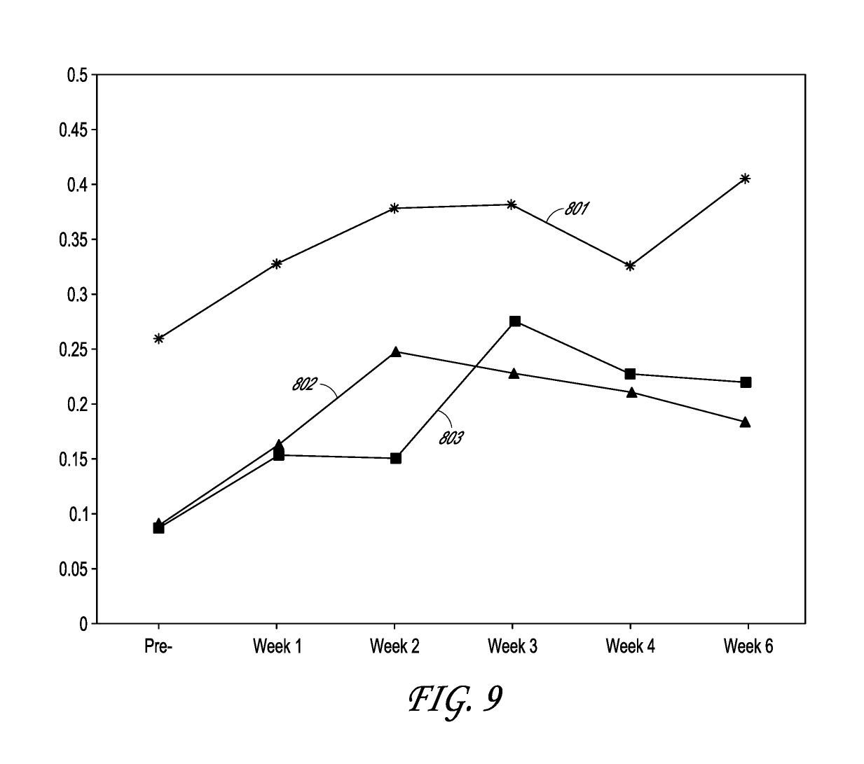

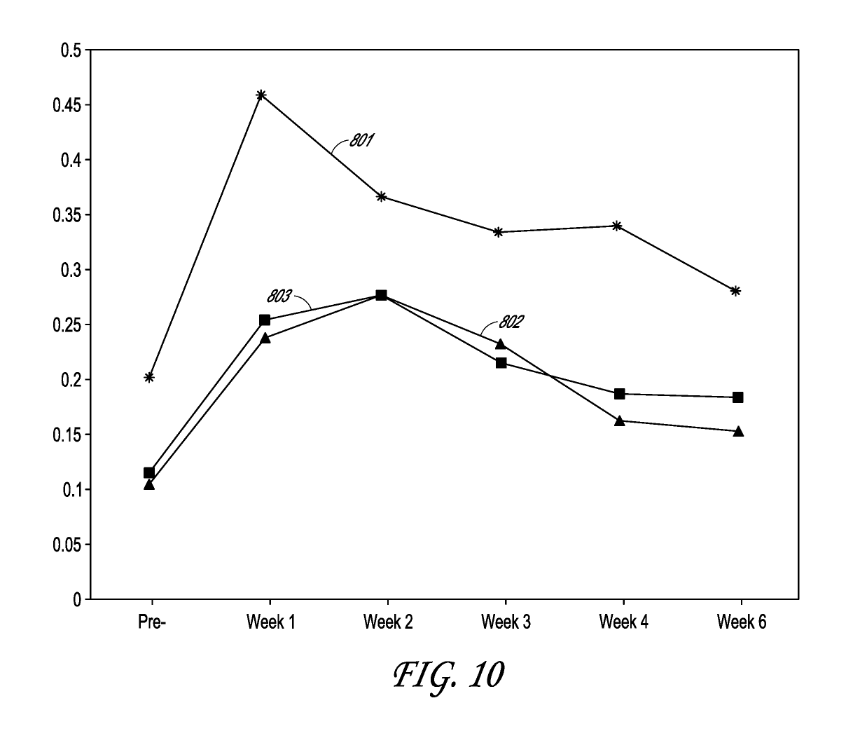

FIG. 8 illustrates the resulting antibody titers as a result of subcutaneous polyvalent vaccination with 1 μg of Streptococcus pneumoniae polysaccharide antigen from Example 3. The antibody titers here and in FIG. 9 and FIG. 10 were tested against Streptococcus polysaccharide conjugated to ELISA plates.

This figure plots absorbance on the y-axis versus time on the x-axis. The absorbance directly relates to antibody titer in mouse serum. The antibody titers of the control (unsplenectomized) group are illustrated as line 801. Similarly, line 802 illustrates the splenectomized group where splenic lymphocytes were reinfused. Line 803 meanwhile illustrates the antibody titers of the splenectomized group with no reinfusion of splenic lymphocytes.

In all groups, peak antibody titers were reached in the period around one week after immunization. Nonsplenectomized animals, illustrated in line 801, demonstrated higher antibody titers in reaction to the Streptococcus pneumoniae vaccination compared to the splenectomized mice in lines 802 and 803. The splenectomized group with splenic lymphocyte reinfusion, in line 802, had an elevated immune response at one week compared with the group without splenic lymphocytes.

Without wishing to be bound by theory, it is believed that the reinfused splenic lymphocytes provided to the splenectomized group strengthened the humoral immune response, as the spleen may serve as a repository for certain subsets of lymphocytes, for example B cells, that may be reactive to antigens such as polysaccharide antigens. Here, the splenectomized group receiving a reinfusion of splenic lymphocytes exhibited a greater immune response, in particular of the humoral immune response, compared to splenectomized mice not receiving a reinfusion of splenic lymphocytes.

Of course, other antigens may be administered to increase the immunity after splenectomy. Vaccines, in particular polysaccharide or protein conjugated vaccines, may be administered. Some vaccines that may be administered to increase immunity after splenectomy include without limitation vaccines against Haemophilus influenzae, Streptococcus pneumoniae, and Neisseria meningitides. Although the antigen in this example was administered subcutaneously, other administration routes are also possible, including via intramuscular, intravenous, oral, and other such administration routes.

In some embodiments, a biological response modifiers (BRMs) may be administered. In some embodiments, BRMs may be administered in combination with splenocytes and/or splenic lymphocytes. In some embodiments, a BRM can be OK-432.

FIG. 9 illustrates the basal antibody titer of three groups of mice where no antigen was administered. As before, the unsplenectomized control group is illustrated as line 801, while the splenectomized group with reinfused lymphocytes is illustrated as line 802. The splenectomized group with no reinfusion is illustrated as line 803.

In this figure, while the unsplenectomized group shows a higher basal antibody titer against Streptococcus pneumoniae, the splenectomized group which underwent splenic lymphocyte reinfusion (line 802) showed an elevated antibody titer compared to the group in line 803 for the first two weeks, after which the antibody titers become similar.

This figure also indicates that intact spleens result in a higher basal antibody secretion against Streptococcus pneumoniae, most likely due to the spleen harboring a subpopulation of cells responsible for recognizing this antigen. Accordingly, improved methods of readministering cells extracted from splenic tissue may improve humoral immunity after splenectomy.

FIG. 10 illustrates the antibody titer of three groups of mice which were intravenously administered with killed Streptococcus pneumoniae Type III cells. The lines denote the same groups used in the previous figures. Here, the unsplenectomized control group exhibits an elevated antibody response at one week. Both of the splenectomized groups (with and without reinfused splenic lymphocytes) show a lower, yet somewhat elevated, response peaking approximately two weeks after administration of killed cells.

Without wishing to be bound by theory, these results indicate the possibility of a more involved mechanism of response towards T-dependent antigens (e.g., bacteria, virus-infected cells, tumor cells) not easily rectified by a reinfusion of lymphocytes. Increased immunity may involve both the structure of the spleen in conjunction with the reticuloendothelial system, or a lack of pre-existing antibodies that may be used to opsonize the killed cells. However, studies have shown that the spleen is responsible for only a small portion of the clearance for T-dependent antigens such as bacteria compared to the liver and other organs. As such, post-splenectomy cellular immunity may be improved by immunization prior to the splenectomy, as antibodies against T-dependent antigens would then permit such antigens to be cleared without the spleen.

Accordingly, the cell-mediated immunity after splenectomy may be increased by reinfusion of lymphocytes that had been previously exposed or challenged to a T-dependent antigen. For example, the immune response in FIG. 10 for the splenectomized mice receiving a lymphocyte reinfusion may be increased if the mice had received a prior vaccination or exposure to killed Streptococcus pneumoniae cells prior to splenectomy. In some embodiments, the cell-mediated immunity after splenectomy may be increased by reinfusion of splenocytes in combination with a BRM.

Further, immunity—in particular cell-mediated immunity—against tumor or cancerous cells, as well as non-cancerous tumors, can also be increased by reinfusion of splenocytes and splenic lymphocytes. In some embodiments, immunity—in particular cell-mediated immunity—against tumor or cancerous cells may also be increased by reinfusion of splenocytes and splenic lymphocytes.

It is believed that there is a population of splenic cells that may be responsible for additional protection against tumor cells. For example, one study has identified that certain splenic cells may react to a Streptococcal preparation (OK-432) may identify and destroy liver tumor cells when a spleen is present. The reinfusion of splenic lymphocytes may thus boost immune response to tumor or cancerous cells after splenectomy.

Thus, in some embodiments, a method of potentiating an immune response against a cancer or a non-cancerous tumor or a combination thereof in a patient in need thereof by removing all or a part of the patient's spleen, harvesting splenocytes from the removed all or part of the patient's spleen and administering the harvested splenocytes to the patient is provided. In some embodiments, the splenocytes are obtained for this purpose as described hereinabove.

In some embodiments, the method comprises removing all or a part of the patient's spleen. In some embodiments, a part of the patient's spleen can be about 0.1% to 99.9% of the patient's spleen. In some embodiments, the method comprises removing 100% of the patient's spleen.

In some embodiments, the method comprises harvesting splenocytes from the removed all or part of the patient's spleen. In some embodiments, the number of splenocytes harvested can range from about 10 million cells per gram of tissue to about 10 million cells per gram of tissue.[0088] In some embodiments, the method comprises administering the harvested splenocytes to the patient. In some embodiments, the number of splenocytes administered ranges from about 5 trillion to about 100 trillion cells. In some embodiments, the volume in which the harvested splenocytes are administered is about 250 ml to about 1000.

In some embodiments, administering the harvested splenocytes potentiates the immune response against the cancer or the tumor or the combination thereof. In some embodiments, the immune response is cell mediated, humoral or both. In some embodiments, humoral immunity is potentiated. In some embodiments, both cell mediated immunity and humoral immunity are potentiated.

In some embodiments, both cell mediated immunity and humoral immunity are potentiated but cell mediated immunity is potentiated more than humoral immunity. In some embodiments, both cell mediated immunity and humoral immunity are potentiated but humoral immunity is potentiated more than cell mediated immunity. In some embodiments, both cell mediated immunity and humoral immunity are potentiated by a similar amount.

In some embodiments, the immune response against the cancer or the non-cancerous tumor or the combination thereof is potentiated as measured by an increase in a cellular immunity, a humoral immunity or both against the cancer or the non-cancerous tumor or the combination thereof.

In some embodiments, the patient is administered a biological response modifier (BRM) prior to administering the harvested splenocytes. In some embodiments, the patient is administered the BRM simultaneously with administering the harvested splenocytes. In some embodiments, the patient is administered the BRM after administering the harvested splenocytes.

In some embodiments, BRMs comprise immunomodulatory agent, immunoadjunctive agent or a combination thereof. Non-limiting examples of immunomodulatory agents are interleukins, cytokines, chemokines, and immunomodulatory imide drugs. Non-limiting examples of immunoadjunctive agents are OK-432, polysaccharides, cytokines, and antibodies.

In some embodiments, the patient is administered a biological response modifier (BRM) prior to administering the harvested splenocytes. In some embodiments, the patient is administered the BRM simultaneously with administering the harvested splenocytes.

In some embodiments, the patient's cancer or non-cancerous tumor or both has been removed by surgery. In some embodiments, the patient is additionally administered chemotherapy, radiation therapy or both either before, during or after administration of salvaged splenocytes and splenic lymphocytes

In some embodiments, the BRM is administered orally, subcutaneously, intradermally, intravenously, intramuscularly or intraperitoneal or combination thereof. In some embodiments, the harvested splenocytes are administered intravenously.

However, in some embodiments, one or more of the following routes of administration are also contemplated for the administration of the BRM and splenocytes: parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal. In some embodiments, the composition to be administered can be formulated for delivery via one or more of the above noted routes.

In some embodiments, the BRM is an agent that stimulates the patient's immune system against the cancer or, the non-cancerous tumor or the combination thereof.

In some embodiments, the immune response against a cancer or a non-cancerous tumor or a combination thereof has been weakened or destroyed in the patient. In some embodiments, the immune response against a cancer or a non-cancerous tumor or a combination thereof has been weakened or destroyed in the patient by one or more of a chemotherapy, a radiation therapy, a genetic factor, or a co-morbid factor.

Examples of genetic factors would be patient with DiGeorge syndrome who are athymic and these along with other immundeficiencies as well as certain genetic mutations in p53 and other syndromes leave patients at risk of developing certain cancers. Interestingly, patients with congenital heart disease and DiGeorge syndrome also may be asplenic.

In some embodiments, the harvesting comprises homogenizing the part of the patient's spleen and isolating a cellular fraction. In some embodiments, the cellular fraction is preserved for future use. Non-limiting methods by which the cellular fraction can be preserved are cryopreservation, in glycerol, lyophilization.

Non-limiting examples of cells in the cellular fraction are splenocytes, T lymphocytes, B lymphocytes, hematopoietic cells, stem cells, erythrocytes, leukocytes, monocytes, macrophages, natural killer cells, and dendritic cells.

In some embodiments, a composition for potentiating an immune response against a cancer or a non-cancerous tumor or a combination thereof in a patient in need thereof is provided. In some embodiments, the composition comprises splenocytes harvested from all or part of the patient's spleen. In some embodiments, the composition additionally comprises one or more pharmaceutically acceptable carriers. In some embodiments, the composition additionally comprises a BRM, a chemotherapeutic agent or other immunomodulators such as but not limited to monoclonal antibodies, cytokines or other immunomodulating cells, proteins, saccharides or lipids.

In some embodiments, one or more of the following routes of administration are also contemplated for the administration of the composition: oral, subcutaneous, intradermal, intravenous, parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

It should be understood, that this detailed description, while indicating some preferred embodiments, is given by way of illustration only, since various changes and modifications within the spirit and scope of the invention will become apparent to those of ordinary skill in the art. For example, one of ordinary skill in the art can assemble a kit based on the methods and compositions provided herein.

Although this invention has been disclosed in the context of certain embodiments and examples, those skilled in the art will understand that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure.

It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes or embodiments of the disclosed invention. Thus, it is intended that the scope of the present invention herein disclosed should not be limited by the particular disclosed embodiments described above.

What is claimed is:

1. A method of potentiating an immune response against a cancer or a non-cancerous tumor or a combination thereof in a patient in need thereof, the method comprising:
removing all or a part of the patient's spleen;
harvesting splenocytes from the removed all or part of the patient's spleen; and
administering the harvested splenocytes to the patient,
thereby potentiating the immune response against the cancer or the tumor or the combination thereof.

2. The method of claim 1, wherein the immune response against a cancer or a non-cancerous tumor or a combination thereof has been weakened or destroyed in the patient.

3. The method of claim 2, wherein the immune response against a cancer or a non-cancerous tumor or a combination thereof has been weakened or destroyed in the patient by one or more of a chemotherapy, a radiation therapy, a genetic factor, or a co-morbid factor.

4. The method of claim 1, wherein the immune response is cell mediated, humoral or both.

5. The method of claim 1, wherein the patient is administered a biological response modifier (BRM) or other immunomodulator or immunotherapy prior to administering the harvested splenocytes, the patient is administered the BRM or other immunomodulator or immunotherapy simultaneously with administering the harvested splenocytes, the patient is administered the BRM or other immunomodulator or immunotherapy after administering the harvested splenocytes, or a combination of the foregoing.

6. The method of claim 5, wherein the BRM is administered orally, subcutaneously, intradermally, intravenously, intramuscularly or intraperitoneal or combination thereof.

7. The method of claim 5, wherein the harvested splenocytes are administered intravenously.

8. The method of claim 5, wherein the BRM is an agent that stimulates the patient's immune system against the cancer, the non-cancerous tumor or the combination thereof.

9. The method of claim 5, wherein the BRM is an immunomodulatory agent or an immunoadjunctive agent.

10. The method of claim 5, wherein the BRM is OK-432.

11. The method of claim 1, wherein the immune response against the cancer or the non-cancerous tumor or the combination thereof is potentiated as measured by an increase in a cellular immunity, a humoral immunity or both against the cancer or the non-cancerous tumor or the combination thereof.

12. The method of claim 1, wherein harvesting comprises homogenizing the part of the patient's spleen and isolating a cellular fraction.

13. The method of claim 12, wherein the cellular fraction is preserved for future use.

14. The method of claim 1, wherein the patient is additionally administered chemotherapy, radiation therapy or other immunotherapeutic treatment or any combination thereof.

15. The method of claim 1, wherein the patient's cancer or non-cancerous tumor or both has been removed by surgery.

16. A device for harvesting cells from all or a part of an organ from a patient, the device comprising:
a top portion comprising a lid and a crank comprising a crankshaft rotatably attached to the lid and extending therethrough in a longitudinal direction, wherein the crankshaft further comprises a grinder attached at a distal end of the crankshaft;
a bottom portion comprising a filtering component at the proximal end of the bottom portion; and
a middle portion defining a cylindrical body, the middle portion configured to be placed between the top portion and the bottom portion, wherein the top, middle, and bottom portions are joined together to define a cavity therein, and wherein the grinder is configured to grind all or a part of the organ from the patient placed within the cavity.

17. The device of claim 16, further comprising a handle attached to the crank.

18. The device of claim 16, wherein the crank is attached to a motor.

19. The device of claim 16, wherein the device comprises a port configured to be connected to a fluid source.

20. The device of claim 19, wherein the lid comprises one or more channels in fluidic communication with the port and the cavity to permit the fluid from the port to enter the cavity.

21. The device of claim 16, wherein the grinder implement comprises a grinding surface provided with one or more cutting channels.

22. The device of claim 16, wherein at least one of the top portion and middle portion, or the middle portion and bottom portion, may be secured together by threads.

23. The device of claim 16, wherein the filtering component comprises a sieve or a membrane filter.

24. The device of claim 16, wherein a distal end of the bottom portion is configured to attach to a collection device.

25. The device of claim 16, wherein the organ is spleen.

26. A method of obtaining a population of splenocytes, the method comprising:
inserting all or part of a spleen into a device for harvesting cells from all or a part of an organ from a patient, the device comprising:
a cavity configured to receive all or a part of the organ, the cavity comprising a grinder implement at a proximal end of the cavity and a filtering component at a distal end of the cavity;
grinding all or a part of the organ by pressing and rotating the grinder implement against the tissue;
supplying fluid into the cavity;
filtering the ground all or a part of the organ and fluid through the filtering component; and
collecting the ground all or a part of the organ and fluid in a receptacle.

27. The method of claim 26, further comprising harvesting splenocytes from the collected all or a part of the organ and fluid.
